(19) United States
(12) Patent Application Publication
(10) Pub. No.: US 2024/0180456 A1
(43) Pub. Date: Jun. 6, 2024

(54) CLIP-ON OPTICAL OR ECG LIGHT BASED PHYSIOLOGICAL MEASUREMENT DEVICE

(71) Applicant: Masimo Corporation, Irvine, CA (US)

(72) Inventors: Ammar Al-Ali, San Juan Capistrano, CA (US); Stephen Scruggs, Newport Beach, CA (US); James Ford Schramm, Huntington Beach, CA (US)

(21) Appl. No.: 18/528,600

(22) Filed: Dec. 4, 2023

Related U.S. Application Data
(60) Provisional application No. 63/386,058, filed on Dec. 5, 2022.

(51) Int. Cl. A61B 5/1455; A61B 5/00

(52) U.S. Cl. CPC A61B 5/14552; A61B 5/6803; A61B 5/6838

(57) ABSTRACT

A light-based or ECG physiological measurement system including a clip-on device configured to secure to a head-worn accessory while still allowing physiological measurement from a patent. The device may include a body; a physiological parameter measurement sensor at least partially disposed inside the body and configured to measure one or more physiological parameters of a user. The physiological parameter measurement sensor includes a securing mechanism configured to attach the physiological parameter measurement sensor to a user wearable device.

### INCORPORATION BY REFERENCE TO ANY PRIORITY APPLICATIONS

The present application claims priority to U.S. Provisional Application No. 63/386,058, filed Dec. 5, 2022. The above-listed application and any and all other applications for which a foreign or domestic priority claim is identified in the Application Data Sheet as filed with the present application, are hereby incorporated by reference under 37 CFR 1.57.

### FIELD

The present disclosure relates to a clip-on device for securing a physiological measurement device to a portion of a user's body and/or wearable accessories.

### BACKGROUND

Physiological measurement devices can collect or analyze a patient's physiological parameters such as blood oxygen saturation level, temperature, respiratory rate, pulse rate, blood pressure, and the like. Such devices can include, for example, acoustic sensors, electroencephalogram (EEG) sensors, electrocardiogram (ECG) devices, blood pressure monitors, temperature sensors, pulse oximeters, among others. The use of physiological measurement devices can be used to diagnose a condition, monitor the efficacy of a treatment, and/or identify potential health risks.

### SUMMARY

There is provided in accordance with one aspect of the present disclosure, an optical or ECG based physiological measurement device. The optical or ECG based physiological measurement device includes a body; a physiological parameter measurement sensor at least partially disposed inside the body and configured to measure one or more physiological parameters of a user, the physiological parameter measurement sensor comprising a light emitter and a light detector, the light emitter emitting light of at least two wavelengths and wherein the physiological parameter measurement sensor is further configured to measure pulse oximetry (SpO2) or ECG; wherein the physiological parameter measurement sensor comprises a securing mechanism configured to attach the physiological parameter measurement sensor to a user wearable device.

There is provided in accordance with one aspect of the present disclosure a clip-on device configured to secure to a head-worn accessory. The clip-on device includes a body; a physiological parameter measurement sensor at least partially disposed inside the body and configured to measure one or more physiological parameters of a user; wherein the physiological parameter measurement sensor includes a securing mechanism configured to attach the physiological parameter measurement sensor to a user wearable device.

In some aspects, the securing mechanism includes a channel extending along a length of the clip-on device, the channel configured to receive and secure at least a portion of the user wearable device.

In some aspects, a size of the channel can expand or contract as the portion of the user wearable device is inserted in the channel.

In some aspects, the securing mechanism includes a first panel including a first end, a second end opposite the first end; a second panel including a first end, and a second end opposite the first end; and a hinge configured to secure the first panel and the second panel together and transition the clip-on device between a first position, a second position, and a third position; wherein when the clip-on device is in the first position, the first end of the first panel and the first end of the second panel are not in contact forming a first slit, and the second end of the first panel and the second end of the second panel are not in contact forming a second slit; wherein the first and second slits can receive a portion of the user wearable device; wherein when the clip-on device is in the second position, the first end of the first panel and the first end of the second panel are in contact forming a first aperture, wherein the first aperture can secure the portion of the user wearable device; and wherein when the clip-on device is in the third position, the second end of the first panel and the second end of the second panel are in contact forming a second aperture, wherein the second aperture can secure the portion of the user wearable device.

In some aspects, the first panel and a shape of the second panel are the same.

In some aspects, the first aperture and the second aperture extend a length of the clip-on device.

In some aspects, the first aperture and the second aperture extend a width of the clip-on device.

In some aspects, the user wearable device includes a pair of sunglasses.

In some aspects, the user wearable device includes a pair of eyeglasses.

In some aspects, the user wearable device includes a pair of bone conduction headphones.

In some aspects, the one or more physiological parameters include at least one of oxygen saturation and pulse rate of the user.

In some aspects, the physiological parameter measurement sensor includes at least one emitter and at least one detector.

In some aspects, the at least one emitter and the at least one detector contact at least a portion of the user's body when the clip-on device is secured to the user wearable device and the user wearable device is being worn by the user.

In some aspects, the portion of the user's body includes a head of the user.

There is provided in accordance with another aspect of the present disclosure a physiological measurement device. The physiological measurement device includes a main body including, a first portion including a first face and a second face, a second portion including a first face and a second face, and a hinge portion positioned between the first portion and the second portion, the hinge portion mechanically coupling the first portion and the second portion; a physiological parameter measurement sensor configured to measure one or more physiological parameters of a user, the physiological parameter measurement sensor including at least one emitter and at least one detector, the at least one emitter and the at least one detector positioned on the first face of the first portion; wherein the first portion and the second portion are configured to form at least one channel capable of receiving and securing a user wearable device.

In some aspects, the first portion further includes a first end, and a second end opposite the first end; the second portion further includes a first end, and a second end opposite the first end; and the hinge portion configured to transition the main body between at least a first position, a second position, and a third position.

In some aspects, when the main body is in the first position, the first end of the first portion and the first end of the second portion are not in contact forming a first aperture, and the second end of the first portion and the second end of the second portion are not in contact forming a second aperture; wherein the first and second apertures can receive a portion of the user wearable device.

In some aspects, when the main body is in the second position, the first end of the first portion and the first end of the second portion are in contact forming a first channel, wherein the first channel can secure the portion of the user wearable device.

In some aspects, when the main body is in the third position, the second end of the first portion and the second end of the second portion are in contact forming a second channel, wherein the second channel can secure the portion of the user wearable device.

In some aspects, a shape of the first portion and a shape of the second portion are the same.

In some aspects, the first aperture and the second aperture extend a length of the main body.

In some aspects, the first aperture and the second aperture extend a width of the main body.

In some aspects, the user wearable device includes a pair of sunglasses.

In some aspects, the user wearable device includes a pair of eyeglasses.

In some aspects, the user wearable device includes a pair of bone conduction headphones.

In some aspects, the one or more physiological parameters include at least one of oxygen saturation and pulse rate of the user.

In some aspects, the at least one detector includes a near field detector and a far-field detector.

In some aspects, the at least one emitter is configured to generate and emit a near-field optical path and a far-field optical path through a tissue site of the user, wherein the near field detector is configured to detect the near-field optical path after attenuation through the tissue site of the user, and wherein the far field detector is configured to detect the far-field optical path after attenuation through the tissue site of the user.

### BRIEF DESCRIPTION OF THE DRAWINGS

FIGS. 1 and 2 show an example of a physiological measurement device.

FIGS. 3-5 show the physiological measurement device shown in FIGS. 1 and 2 in contact with the skin of a user.

FIGS. 6-8 show the physiological measurement device shown in FIGS. 1 and 2 attached to different positions of a pair of bone conduction headphones.

FIG. 9 shows the physiological measurement device shown in FIGS. 1 and 2 attached to a pair of sunglasses.

FIG. 10 shows the physiological measurement device shown in FIGS. 1 and 2 with a near-field and a far-field emitter to detector optical paths.

### DETAILED DESCRIPTION

Although certain embodiments and examples are described below, those of skill in the art will appreciate that the disclosure extends beyond the specifically disclosed embodiments and/or uses and obvious modifications and equivalents thereof based on the disclosure herein. Thus, it is intended that the scope of the disclosure herein disclosed should not be limited by any particular embodiments described below.

Daily use of a physiological measurement device can be beneficial to a user (also referred to as a “wearer” herein). For instance, physiological measurement devices which incorporate pulse oximetry components can be utilized to measure and/or monitor various physiological parameters and/or characteristics such as oxygen saturation (SpO2), pulse rate, a plethysmograph waveform, perfusion index (PI), pleth variability index (PVI), methemoglobin (MetHb), carboxyhemoglobin (CoHb), total hemoglobin (tHb), glucose, electrocardiogram (ECG) parameters, among others. The various physiological parameters and/or characteristics can beneficially be used to diagnose a condition, monitor the efficacy of a treatment, and/or identify potential health risks.

A physiological measurement device as described herein can include a clip-on device. The clip-on device can include a securing mechanism for securing the physiological measurement device to a portion of a user's body and/or to a wearable device. For instance, the securing mechanism of the physiological measurement device can be secured to sunglasses, eyeglasses, headphones, earphones, bone conduction headphones, and/or any other wearable device and/or item. This can allow users to integrate the physiological measurement device easily and conveniently into more than one wearable device and/or item. The physiological measurement device can also be swapped from one wearable device and/or item to another.

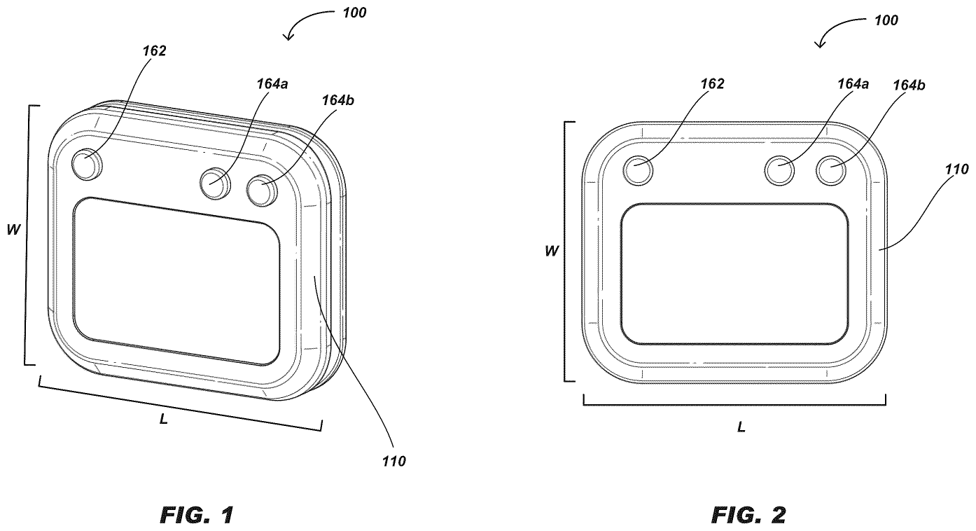

As shown in FIG. 1, the physiological measurement device 100 (also referred herein to as a main body or housing) can include a physiological parameter measurement module configured to measure an indication of the wearer's physiological parameters and/or characteristics, which can include, for example, pulse rate, respiration rate, SpO2, Pleth Variability Index (PVI), Perfusion Index (PI), Respiration from the pleth (RRp), hydration, and/or other parameters and/or characteristics. The physiological parameter measurement module can include a skin-interfacing cover that encloses the one or more plurality of emitters 162 (such as LEDs) and the one or more detectors 164a, 164b (such as photodiodes).

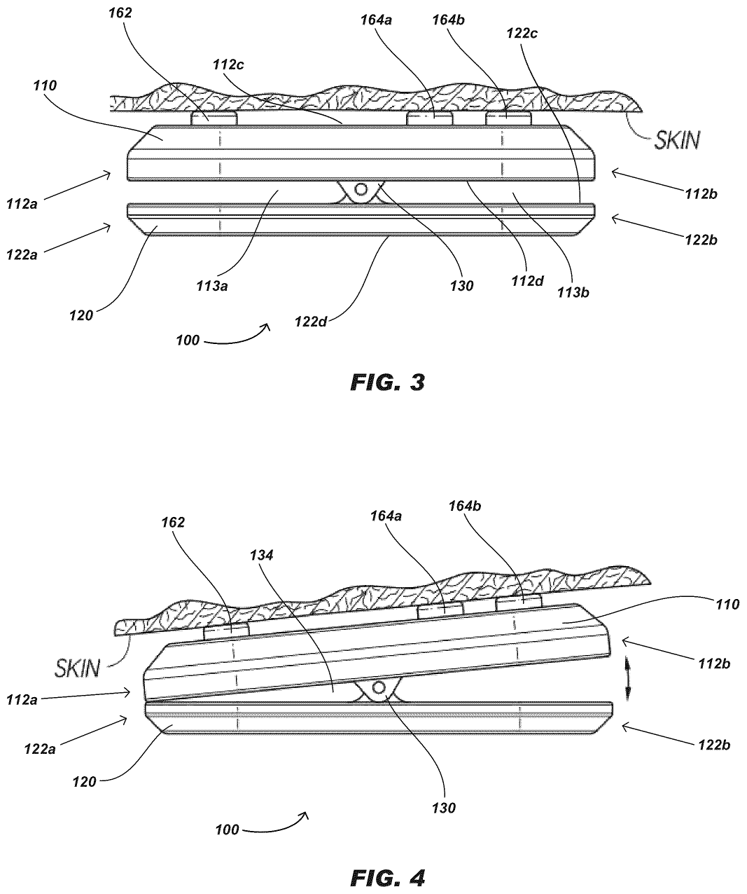

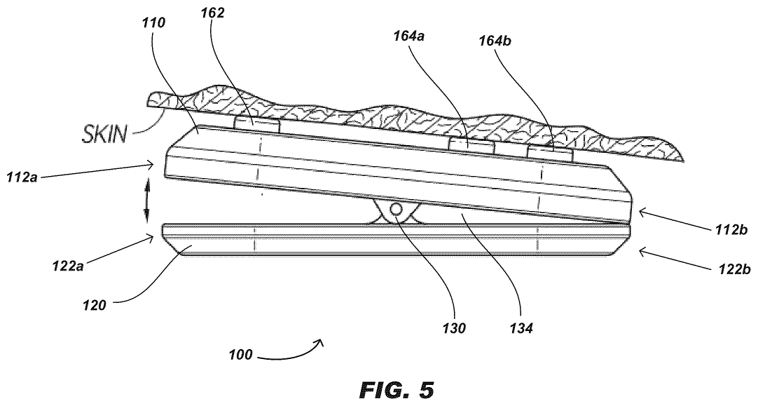

FIGS. 1-2 illustrate top perspective and top views of a physiological measurement device 100. FIGS. 3-5 illustrate a physiological measurement device 100 in contact with a skin of a user. The physiological measurement device 100 can include a securing mechanism for attaching the physiological measurement device 100 to another device and/or item. The device and/or item can include, but is not limited to, sunglasses, eyeglasses, headphones, earphones, bone conduction headphones, and/or any other wearable device and/or item. In some cases, the physiological measurement device 100 can be attached to and detached from devices as many times as needed thereby allowing users to use the physiological measurement device 100 with as many devices as desired. For example, the physiological measurement device 100 can be secured to a pair of sunglasses using the securing mechanism. The physiological measurement device 100 can then be detached from the sunglasses and reattached to a pair of bone conduction headphones. Beneficially, this provides a convenient, yet inexpensive, way for users to add physiological measurement capabilities to a wide variety of devices and/or items.

In some cases, the physiological measurement device 100 can be integrated into a device and/or item. For example, the physiological measurement device 100 can be part of the construction of the device and/or item. Using a pair of eyeglasses as an example, the physiological measurement device 100 can be integrated into one of the temples. Users can add physiological measuring capabilities to exiting devices and/or items, such as a pair of eyeglasses simply by replacing at least one of the stems of the eyeglasses with a stem incorporating a physiological measurement device 100.

The physiological measurement device 100 can include a first panel 110 and a second panel 120 (also referred herein to as first portion and second portion respectively). The first panel 110 can include a first end 112a and a second end 112b. The first panel 110 can also include a first face 112c and a second face 112d. Similarly, the second panel 120 can include a first end 122a and a second end 122b. The second panel 120 can also include a first face 122c and a second face 122d. The first and second panels 110, 120 can be coupled to each other via a hinge 130, as shown in FIGS. 3-5. In some cases, the first and second panels 110, 120 can be coupled to each using a screw and/or magnets. The hinge 130 can be configured to allow the physiological measurement device 100 to transition between at least three positions. In a first position, and as shown in FIG. 3, the first and second ends 112a, 112b of the first panel 110 are not in contact with the first and second ends 122a, 122b of the second panel 120. In a second position, and as shown in FIG. 4, the first ends 112a, 122a are in contact with each other and second ends 112b, 122b are not in contact with each other. Lastly, in a third position, and as shown in FIG. 5, the first ends 112a, 122a are not in contact with each other and the second ends 112b, 122b are in contact with each other. A user can transition the physiological measurement device 100 from one state to another state by, for example bringing first ends 112a, 122a together, bringing second ends 112b, 122b together, or by moving the first and second panels 110, 120 so that neither the first ends 112a, 122a or the second ends 112b, 122b are in contact with each other. The physiological measurement device 100 can include one or more emitters 162 (such as LEDs) and one or more detectors 164a, 164b (such as photodiodes). In some cases, the detector 164a includes a near field detector and the detector 164b includes a far-field detector. The one or more emitters 162 and the one or more detectors 164a, 164b can be positioned on the first panel 110, and their position on FIGS. 1 and 2 can be switched. In some cases, the one or more emitters 162 and the one or more detectors 164a, 164b can be positioned on the first face of the 112c of the first panel 110. However, the one or more emitters 162 and the one or more detectors 164a, 164b can be positioned anywhere on the measurement device 100 (e.g., on the second panel 120).

When the physiological measurement device 100 is in the first position, the first ends 112a, 122a define a first slit 113a and the second ends 112b, 122b define a second slit 113b, as shown in FIG. 3. When the physiological measurement device 100 is in the second or third positions, the first ends 112a, 122b and the second ends 112b, 122b can define an aperture. For example, and as shown in FIG. 4, when the first ends 112a, 122a are in contact with each other, a first aperture 124 is defined. Similarly, and as shown in FIG. 5, when the second ends 112b, 122b are in contact with each other, a second aperture 134 is defined. The first sand second apertures 124, 134 can extend across a width W of the physiological measurement device 100. In some cases, the first and second apertures extend across a length L of the physiological measurement device 100.

In some cases, instead of the first panel 110 and a second panel 120 being attached to each other using a hinge, a screw, and/or magnets, the first and second panels 110, 120 can be structurally integral and form a main channel. The main channel can receive at least one portion of a device and/or item. For example, a temple of a pair of sunglasses can be inserted into the main channel. The main channel can have a tight construction thereby allowing it to secure the physiological measurement device 100 to the device and/or item (e.g., stem of a pair of sunglasses) yet preventing undesired and/or accidental movement of the physiological measurement device 100 along the structure of the device and/or item. For example, the tight construction of the main channel can allow users to insert and/or slide a portion of the device and/or item into the main channel and to adjust the position of the physiological measurement device 100 along the device and/or item. At the same time, the tight construction of the main channel can prevent the physiological measurement device from moving unless a minimum threshold force is applied (e.g., unless a user moves the physiological measurement device 100 and/or the device).

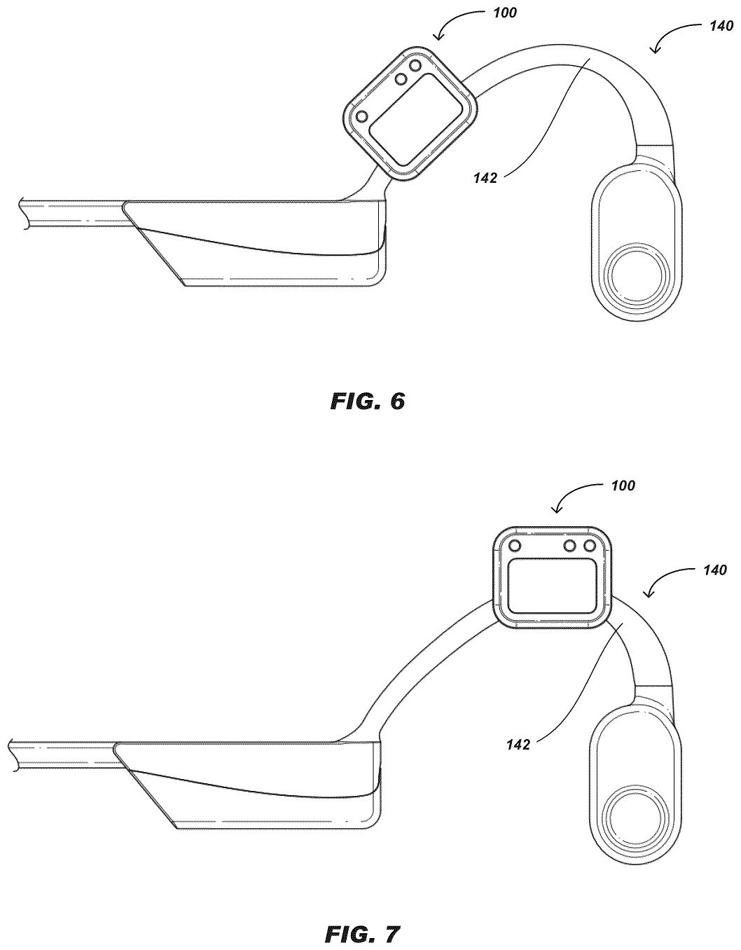

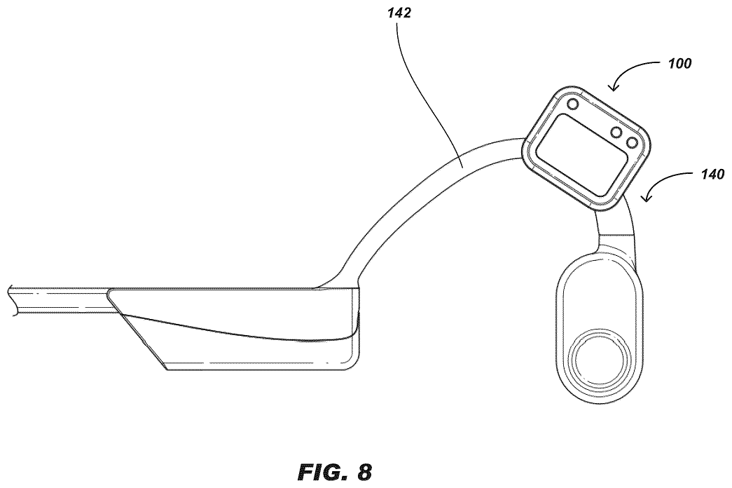

As shown in FIGS. 6-9, the physiological measurement device 100 can be attached to other devices or artifacts such as sunglasses, eyeglasses, headphones, or bone conduction headphones, etc. For example, the physiological measurement device 100 can be attached to a pair of bone conduction headphones 140, as shown in FIGS. 6-8. The apertures 124, 134 can facilitate securing of the physiological measurement device 100 to the bone conduction headphones 140. The physiological measurement device 100 can be attached to the bone conduction headphones 140 by inserting a portion of a temple 142 of the bone conduction headphones 140 into the first or second slits 113a, 113b. After inserting a portion of the temple 142 into the first or second slits 113a, 133b, the physiological measurement device 100 can be transitioned to the second or third states thereby securing a portion of the temple 142 within apertures 124 or 134. The physiological measurement device 100 can be secured along any portion of the temple 142, as shown by FIGS. 6-8. When the physiological measurement device 100 is attached to the bone conduction headphones 140, the one or more of a plurality of emitters 162 and the one or more detectors 164a, 164b can be in contact with a skin of a user, as shown in FIGS. 3-5. The hinge 130 can be configured to pivot to allow the one or more of a plurality of emitters 162 and the one or more detectors 164a, 164b to maintain constant contact with the skin of a user. Beneficially, this can improve the amount of pressure and/or contact applied by the physiological measurement device 100 to the skin of the user when the physiological measurement device 100 is attached to the bone conduction headphones 140.

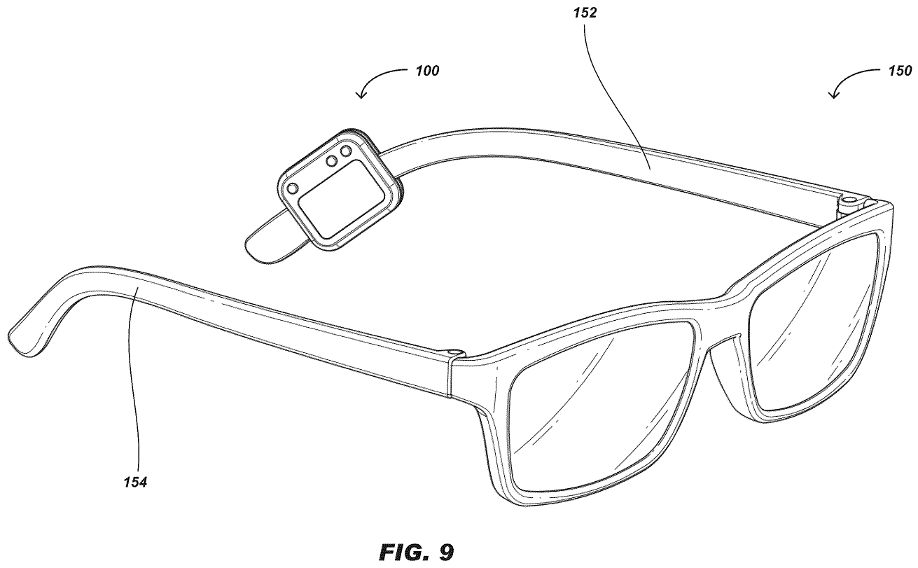

The physiological measurement device 100 can also be attached to a pair of glasses, as shown in FIG. 9. The apertures 124, 134 can facilitate securing of the physiological measurement device 100 to the pair of glasses 150. The physiological measurement device 100 can be attached to the pair of glasses 150 by inserting a portion of temple 152 or temple 154 of the pair of glasses 150 into the first or second slits 113a, 113b. After inserting a portion of temple 152 or temple 154 into the first or second slits 113a, 133b, the physiological measurement device 100 can be transitioned to the second or third states thereby securing a portion of temple 152 or temple 154 within apertures 124 or 134. The physiological measurement device 100 can be secured along any portion of the temples 152, 154. When the physiological measurement device 100 is attached to the pair of glasses 150, the one or more of a plurality of emitters 162 and the one or more detectors 164a, 164b can be in contact with a skin of a user, as shown in FIGS. 3-5. The hinge 130 can be configured to pivot to allow the one or more emitters 162 and the one or more detectors 164a, 164b to maintain constant contact with the skin of a user. Beneficially, this can improve the amount of pressure and/or contact applied by the physiological measurement device 100 to the skin of the user when the physiological measurement device 100 is attached to the bone conduction headphones 140. Even though the physiological measurement device 100 can be attached to another device and/or item as described herein, the physiological measurement device 100 can also be attached to another device and/or item by any suitable means. Suitable means can include, but is not limited to, springs, clamps, clips, wire, wire-ties, ratchets, adhesives, Velcro hook-loop fasteners, tapes, magnets, and/or any other suitable attachment method.

In some implementations, the physiological measurement device 100 includes a module processor (which can include a memory) for driving the emitter(s) 162 to emit light of different wavelengths and/or to process one or more signals responsive to attenuated light after absorption by the body tissue of the wearer from the detectors 164a, 164b. The physiological monitoring device 100 can include various combinations of emitters 162 and/or detectors 164a, 164b. For example, the physiological measurement device 100 can include one emitter 162 and one or more detectors 164a, 164b, two emitters 162 of the same or different wavelengths and one or more detectors 164a, 164b, three or more emitters 162 of the same and/or different wavelengths and one or more detectors 164a, 164b. The physiological measurement device 100 can also include a plurality of detectors 164a, 164b surrounding one or more emitters 162. Optionally, the module processor can also determine and output for display the physiological parameters based on the detected signals. In some cases, the physiological measurement device can include a display (not shown) for displaying the physiological parameters based on the detected signals. The display can be located anywhere of the physiological measurement device 100, like, for example, the first panel 110 and/or the second panel 120. Optionally or alternatively, the physiological measurement device 100 can wirelessly communicate with a user device (e.g., a phone, smart watch, computer, tablet, etc.) that displays the physiological measurements. Alternatively, the physiological measurement device 100 can send the signals from the detectors 164a, 164b (for example, preprocessed signals) to a device processor, which can determine and output for display the physiological parameters based on the detected signals. The absorption of light can be via reflectance and/or transflectance by the wearer's body tissue, for example, by the pulsatile arterial blood flowing within a tissue site where the physiological measurement device 100 is worn (for example, behind the ear).

The emitter(s) 162 of the physiological measurement device 100 can be configured to emit a plurality of (for example, three, four, or more) wavelengths. The emitters 162 can be configured to emit light of a first wavelength providing an intensity signal that can act as a reference signal. The first wavelength can be more absorbent by the human body than light of other wavelengths emitted by the emitters 162. The reference signal can be stronger and less likely to be affected by noise than the signals from other wavelengths emitted by the emitters 162. The reference signal can be used by the module processor to extract information from the other signals, for example, information relevant to and/or indicative of the pulsing rate, harmonics, or otherwise. The module processor can focus the analysis on the extracted information for calculating physiological parameters of the wearer. The first wavelength can be from about 530 nm to about 650 nm, or from about 580 nm to about 585 nm, or from about 6340b nm to about 650 nm, or about 580 nm, or about 6340b nm. The light providing the reference signal can have an orange color. Alternatively, the light providing the reference signal can have a green color.

The emitters 162 can be configured to emit light having a second wavelength having a red color. The second wavelength can be from about 620 nm to about 660 nm. Light of the second wavelength can be more sensitive to changes in oxygen saturation (SpO2). The second wavelength is preferably closer to 620 nm, which results in greater absorption by the body tissue of the wearer, and therefore a stronger signal and/or a stepper curve in the signal, than a wavelength that is closer to 660 nm. The module processor can extract information such as the pleth waveform from signals of the second wavelength.

The emitter(s) 162 can be configured to emit light having a third wavelength of about 900 nm to about 910 nm, or about 905 nm, or about 907 nm. The pulse oximeter processor can use the third wavelength as a normalizing wavelength when calculating ratios of the intensity signals of the other wavelengths.

Additionally or optionally, the emitters 162 can be configured to emit light having a fourth wavelength that is more sensitive to changes in water than the rest of the emitted wavelengths. The fourth wavelength can be about 970 nm. The module processor can determine physiological parameters such as a hydration status of the wearer based at least in part on a comparison of the intensity signals of the fourth wavelength and a different wavelength detected by certain detectors 164a, 164b. The detectors 164a, 164b used for hydration monitoring, which will be described in greater detail below, can be located a predetermined distance away from the emitters 162 so that light travels through a certain depth of the tissue before being detected by those detectors 164a, 164b.

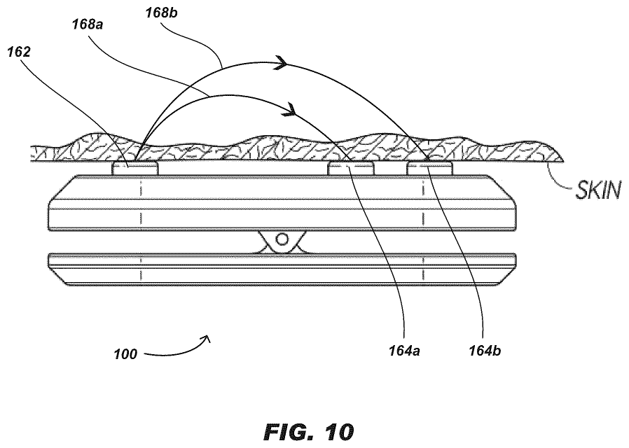

The physiological measurement device 100 can be used to measure and/or monitor various physiological parameters and/or characteristics of a user at or near the head/brain area. FIG. 10 illustrates the physiological measurement device 100 in contact with the skin of a user and generating near-field 168a and far-field 168b emitter-to-detector optical paths through the tissue site of the user. The emitters 162 can generate the near-field 168a and far-field 168b optical paths. The near-field 168a and far-field 168b optical paths can be detected by the detectors 164a, 164b. The resulting detector signals can be processed to calculate and display oxygen saturation (SpO2), delta oxygen saturation (ASpO2) and regional oxygen saturation (rSO2). The emitters 162 and the detectors 164a, 164b can press against the skin of the user, advantageously maximizing the optical transmission and reception of the emitter 162 and detectors 164a, 164b.

The physiological measurement device 100 can optionally include one or more thermistors or other types of temperature sensors. The thermistor(s) can be placed near one or more groups of emitters 162. The thermistor(s) can provide for wavelength correction of the light emitted by the emitters 162. Optionally, the thermistor(s) can additionally measure a temperature of the wearer of the physiological measurement device 100. Optionally there can be one or more thermistors located at other places of the physiological measurement device 100. The physiological measurement device 100 can include a gyroscope, an accelerometer, and/or other position and/or posture detection sensor(s). Optionally, the module processor, the gyroscope, and/or the accelerometer can be located on a printed circuit board (PCB). The emitters 162, the thermistor(s), and/or the detectors 164a, 164b can also be positioned on the PCB in some implementations.

The physiological measurement can include its own device processor, which can be a digital/analog chip or other processor(s), such as a digital watch processor or a smartwatch processor. The physiological measurement device 100 can include a power source, which can be a battery, for powering the device processor, and/or the emitter(s) 162 and/or detector(s) 164a, 164b. The power source can be charged and recharged using a battery connection. The battery connection (not shown) can be positioned on the first panel 110 or the second panel 120.

In some implementations, the physiological measurement device 100 includes a communication module. The communication module can facilitate communication (via wires and/or wireless connection) between the physiological measurement device 100 (and/or components thereof) and separate devices, such as separate monitoring and/or mobile devices. For example, the communication module can be configured to allow the physiological measurement device 100 to wirelessly communicate with other devices, systems, and/or networks over any of a variety of communication protocols. The communication module can be configured to use any of a variety of wireless communication protocols, such as Wi-Fi (802.11x), Bluetooth®, ZigBee®, Z-wave®, cellular telephony, infrared, near-field communications (NFC), RFID, satellite transmission, proprietary protocols, combinations of the same, and the like. The communication module can allow data and/or instructions to be transmitted and/or received to and/or from the physiological measurement device 100 and separate computing devices. The communication module can be configured to transmit (for example, wirelessly) processed and/or unprocessed physiological or other information to a separate computing devices, which can include, among others, a mobile device (for example, an iOS or Android enabled smartphone, tablet, laptop), a desktop computer, a server or other computing or processing device for display and/or further processing, among other things. Such separate computing devices can be configured to store and/or further process the received physiological and/or other information, to display information indicative of or derived from the received information, and/or to transmit information—including displays, alarms, alerts, and notifications—to various other types of computing devices and/or systems that may be associated with a hospital, a caregiver (for example, a primary care provider), and/or a user (for example, an employer, a school, friends, family) that have permission to access the subject's data. As another example, the communication module can be configured to wirelessly transmit processed and/or unprocessed obtained physiological information and/or other information (for example, motion and/or location data) to a mobile phone which can include one or more hardware processors configured to execute an application that generates a graphical user interface displaying information representative of the processed or unprocessed physiological and/or other information. The communication module can be and/or include a wireless transceiver.

### Additional Considerations and Terminology

Although this disclosure has been described in the context of certain examples, it will be understood by those skilled in the art that the present disclosure extends beyond the specifically disclosed examples to other alternative examples and/or uses of the disclosure and obvious modifications and equivalents thereof. In addition, while a number of variations of the disclosure have been shown and described in detail, other modifications, which are within the scope of this disclosure, will be readily apparent to those of skill in the art based upon this disclosure. It is also contemplated that various combinations or sub-combinations of the specific features and aspects of the examples may be made and still fall within the scope of the disclosure. Accordingly, it should be understood that various features and aspects of the disclosure can be combined with or substituted for one another in order to form varying modes of the disclosed.

Features, materials, characteristics, or groups described in conjunction with a particular aspect, or example are to be understood to be applicable to any other aspect, or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples of devices or systems. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that the actual steps taken in the processes illustrated and/or disclosed may differ from those shown in the figures. Depending on the system, certain of the steps described above may be removed, others may be added. Furthermore, the features and attributes of the specific examples disclosed above may be combined in different ways to form additional examples of systems, all of which fall within the scope of the present disclosure. Also, the separation of various system components in the implementations described above should not be understood as requiring such separation in all implementations, and it should be understood that the described components and systems can generally be integrated together in a single product or packaged into multiple products.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more embodiments necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees, 5 degrees, 3 degrees, or 1 degree. As another example, in certain embodiments, the terms “generally perpendicular” and “substantially perpendicular” refer to a value, amount, or characteristic that departs from exactly perpendicular by less than or equal to 10 degrees, 5 degrees, 3 degrees, or 1 degree.

While the above detailed description has shown, described, and pointed out novel features, it can be understood that various omissions, substitutions, and changes in the form and details of the devices or systems illustrated can be made without departing from the spirit of the disclosure. As can be recognized, certain portions of the description herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. An optical or ECG based physiological measurement device comprising:
a body;
a physiological parameter measurement sensor at least partially disposed inside the body and configured to measure one or more physiological parameters of a user, the physiological parameter measurement sensor comprising a light emitter and a light detector, the light emitter emitting light of at least two wavelengths and wherein the physiological parameter measurement sensor is further configured to measure pulse oximetry (SpO2) or ECG;
wherein the physiological parameter measurement sensor comprises a securing mechanism configured to attach the physiological parameter measurement sensor to a user wearable device.

2. A clip-on device configured to secure to a head-worn accessory, the clip-on device comprising:
a body;
a physiological parameter measurement sensor at least partially disposed inside the body and configured to measure one or more physiological parameters of a user;
wherein the physiological parameter measurement sensor comprises a securing mechanism configured to attach the physiological parameter measurement sensor to a user wearable device.

3. The clip-on device of claim 2, wherein the securing mechanism comprises a channel extending along a length of the clip-on device, the channel configured to receive and secure at least a portion of the user wearable device.

4. The clip-on device of claim 3, wherein a size of the channel can expand or contract as the portion of the user wearable device is inserted in the channel.

5. The clip-on device of claim 2, wherein the securing mechanism comprises:
a first panel comprising a first end, a second end opposite the first end;
a second panel comprising a first end, and a second end opposite the first end; and
a hinge configured to secure the first panel and the second panel together and transition the clip-on device between a first position, a second position, and a third position;
wherein when the clip-on device is in the first position, the first end of the first panel and the first end of the second panel are not in contact forming a first slit, and the second end of the first panel and the second end of the second panel are not in contact forming a second slit; wherein the first slit and the second slit can receive a portion of the user wearable device;
wherein when the clip-on device is in the second position, the first end of the first panel and the first end of the second panel are in contact forming a first aperture, wherein the first aperture can secure the portion of the user wearable device; and
wherein when the clip-on device is in the third position, the second end of the first panel and the second end of the second panel are in contact forming a second aperture, wherein the second aperture can secure the portion of the user wearable device.

6. The clip-on device of claim 5, wherein a shape of the first panel and a shape of the second panel are the same.

7. The clip-on device of claim 5, wherein the first aperture and the second aperture extend a length of the clip-on device.

8. The clip-on device of claim 5, wherein the first aperture and the second aperture extend a width of the clip-on device.

9. The clip-on device of claim 2, wherein the user wearable device comprises a pair of sunglasses.

10. The clip-on device of claim 2, wherein the user wearable device comprises a pair of eyeglasses.

11. The clip-on device of claim 2, wherein the user wearable device comprises a pair of bone conduction audio device.

12. The clip-on device of claim 2, wherein the one or more physiological parameters comprise at least one of oxygen saturation and pulse rate of the user.

13. The clip-on device of claim 2, wherein the physiological parameter measurement sensor comprises at least one emitter and at least one detector.

14. The clip-on device of claim 13, wherein the at least one emitter and the at least one detector are configured to contact at least a portion of a user's body when the clip-on device is secured to the user wearable device and the user wearable device is being worn by the user.

15. The clip-on device of claim 14, wherein the portion of the user's body comprises a head of the user.

16. A physiological measurement device comprising:
a main body comprising,
a first portion comprising a first face and a second face,
a second portion comprising a first face and a second face, and
a hinge portion positioned between the first portion and the second portion, the hinge portion mechanically coupling the first portion and the second portion;
a physiological parameter measurement sensor configured to measure one or more physiological parameters of a user, the physiological parameter measurement sensor comprising at least one emitter and at least one detector, the at least one emitter and the at least one detector positioned on the first face of the first portion;
wherein the first portion and the second portion are configured to form at least one channel capable of receiving and securing a portion of a user wearable device.

17. The physiological measurement device of claim 16, wherein:
the first portion further comprises a first end, and a second end opposite the first end;
the second portion further comprises a first end, and a second end opposite the first end; and
the hinge portion configured to transition the main body between at least a first position, a second position, and a third position.

18. The physiological measurement device of claim 17, wherein when the main body is in the first position, the first end of the first portion and the first end of the second portion are not in contact forming a first aperture, and the second end of the first portion and the second end of the second portion are not in contact forming a second aperture; wherein the first aperture and the second aperture can receive a portion of the user wearable device.

19. The physiological measurement device of claim 17, wherein when the main body is in the second position, the first end of the first portion and the first end of the second portion are in contact forming a first channel, wherein the first channel can secure the portion of the user wearable device.

20. The physiological measurement device of claim 17, wherein when the main body is in the third position, the second end of the first portion and the second end of the second portion are in contact forming a second channel, wherein the second channel can secure the portion of the user wearable device.

21. The physiological measurement device of claim 16, wherein a shape of the first portion and a shape of the second portion are the same.

22. The physiological measurement device of claim 18, wherein the first aperture and the second aperture extend a length of the main body.

23. The physiological measurement device of claim 18, wherein the first aperture and the second aperture extend a width of the main body.

24. The physiological measurement device of claim 16, wherein the user wearable device comprises a pair of sunglasses.

25. The physiological measurement device of claim 16, wherein the user wearable device comprises a pair of eyeglasses.

26. The physiological measurement device of claim 16, wherein the user wearable device comprises a pair of bone conduction audio device.

27. The physiological measurement device of claim 16, wherein the one or more physiological parameters comprise at least one of oxygen saturation and pulse rate of the user.

28. The physiological measurement device of claim 16, wherein the at least one detector comprises a near field detector and a far-field detector.

29. The physiological measurement device of claim 28, wherein the at least one emitter is configured to generate and emit a near-field optical path and a far-field optical path through a tissue site of the user, wherein the near field detector is configured to detect the near-field optical path after attenuation through the tissue site of the user, and wherein the-far field detector is configured to detect the far-field optical path after attenuation through the tissue site of the user.
